(19) United States
(12) Patent Application Publication
(10) Pub. No.: US 2014/0275151 A1
(43) Pub. Date: Sep. 18, 2014

(54) DYE FREE LIQUID THERAPEUTIC SOLUTION

(71) Applicants: Mahesh Sudhakar BHARATI, Yapral Secunderabad Pin (IN); Houphouet Hyacinthe YARABE, Richmond, VA (US)

(72) Inventors: Mahesh Sudhakar BHARATI, Yapral Secunderabad Pin (IN); Houphouet Hyacinthe YARABE, Richmond, VA (US)

(21) Appl. No.: 14/200,553

(22) Filed: Mar. 7, 2014

(30) Foreign Application Priority Data
Mar. 15, 2013 (IN) 765/DEL/2013

(51) Int. Cl. A61K 47/10; A61K 31/167

(52) U.S. Cl. CPC A61K 47/10; A61K 31/167
USPC 514/289; 514/772; 514/629; 514/718; 514/653; 514/570

(57) ABSTRACT

A liquid composition is provided. The composition includes at least one active pharmaceutical ingredient dissolved in a liquid carrier system. The liquid carrier includes polyethylene glycol and a co-solvent selected from the group consisting of: propylene glycol, glycerin, and a sugar alcohol. The composition is essentially dye tree, present as a single-phase at room temperature, and optionally contains water.

### BACKGROUND OF THE INVENTION

There are many therapeutic compositions available for use in the treatment of patient cold symptoms such as pain, fever, congestion, and inflammation. The present invention provides a dye-free therapeutic formulation that is present as a single phase at room temperature.

Patients having difficulty swallowing therapeutic compositions in tablet and/or capsule form may find it preferable to ingest the therapeutic compositions in a liquid dosage form where the therapeutic composition is present in a liquid carrier system. The liquid composition is then delivered to the patient in a measured form (e.g. in a prepackaged single dose package, disposable container, or in a bottle from which the patient or care giver can measure the appropriate dosage).

Formulating liquid therapeutic compositions is challenging in terms of selection of suitable liquid carriers. For example, acetaminophen crystallizes in liquid products when it is stored at decreased temperature. Furthermore, acetaminophen degrades at elevated temperatures and produces discolored liquid compositions. These instabilities, inter alia, decrease shelf life of liquid therapeutic compositions and lead to the need to set shorter expiration dates as compared to other-types of dosage forms.

Solutions to these problems can include selecting solvents that are not suitable for many people (e.g. alcohol) and the addition coloring materials, such as dyes, to mask the color of liquid compositions containing degradable active ingredients. Furthermore, many liquid formulations suspend active ingredients in a “two-phase” composition which must be shaken prior to administering a dosage to a patient. These two phase systems generally appear cloudy or opaque in nature. Providing a clear, essentially clear or translucent solution may also provide a benefit in administering the same to patients that are not comfortable ingesting cloudy, opaque or dye-containing medicines.

Thus, there is a need for liquid therapeutic compositions that overcome the problems in the art.

### SUMMARY OF TOE INVENTION

The present invention relates to liquid pharmaceutical compositions suitable for oral administration. The present Inventors have discovered that the liquid therapeutic compositions of the present invention are present in a single phase (e.g. solutions) at room temperature and do not require the addition of coloring material (e.g. dyes) to mask the color of degraded active ingredients (e.g. the actives are stable within the compositions at various storage conditions and durations). The formulations exhibit improved stability and can be present as translucent, and preferably clear solutions.

In one embodiment, a liquid composition is provided. The composition includes an active pharmaceutical ingredient dissolved in a liquid carrier system. The liquid carrier includes a solvent system including polyethylene glycol and a co-solvent selected from the group consisting of propylene glycol, glycerin, and a sugar alcohol. Other co-solvents may include others known to be suitable to one skilled in the art. Thus, the composition can be essentially dye free and present as a single-phase at room temperature.

In a second embodiment, the present invention provides a method of treating a cold and/or cold-like symptoms by administering to an individual a safe and effective amount of the liquid composition described above.

In a third embodiment a method of preparing a liquid formulation is provided.

### DETAILED DESCRIPTION OF THE INVENTION

The present Inventors have discovered polyethylene glycol when melted and mixed with a co-solvent selected from the group consisting of propylene glycol, glycerine, and a sugar alcohol, or the like, serves to solubilize the pharmaceutical compounds and inhibit crystallization at or below room temperature and prevent degradation at or above room temperature. These findings allow for the preparation of liquid compositions which remain in a single phase (e.g. they are not suspensions and instead are solutions) at room temperature. Furthermore, by preventing degradation of the active ingredients, the present inventors have discovered that the addition of coloring materials or agents (e.g. dyes) are not required in the formulation to mask the color of degraded active ingredients.

### Definitions:

“a,” “as,” and “the” as an antecedent refer to either the singular or plural. For example, “an active component” refers to either a single species of compound or a mixture of such species unless the context indicates otherwise.

The phrase “safe and effective amount,” as used herein, is an amount that is effective to mitigate and/or treat the symptoms for which the active ingredient is indicated in a human without undue adverse side effects commensurate with a reasonable risk/benefit ratio. The term “liquid carrier” refers to an solvent system for the active ingredients. The solvent system includes polyethylene glycol and a co-solvent selected from the group consisting of: propylene glycol, glycerin, and a sugar alcohol (or suitable other co-solvents). These composition are preferably essentially free of, or most preferably completely free of, ethanol.

The terms “solvent” and “co-solvent” refer to the non-aqueous portion of the liquid carrier system. The liquid carrier system may optionally contained water, for example up to 50 wt % of the composition.

The term “essentially free of dye” means that the liquid composition does not require a coloring agent to be added to impart color for masking unpleasant color of degraded active. The composition may however contain additional excipients which have natural colors associated therewith. For example additional excipients which may impart color include flavorants (e.g. liquid sugar) or other materials which impart flavor to the composition or non-dye containing excipients that have color.

The term “single phase” is herein understood to mean that the liquid compositions of the present invention are not suspensions at room temperature. The liquid composition(s) of the present invention is a solution at room temperature where the active ingredient(s) remain dissolved in the liquid carrier system. The liquid compositions of the present invention are preferably clear and/or translucent.

Ranges given in the specification are inclusive of either end of the specified range.

Reference throughout the specification to “one embodiment,” “another embodiment,” “an embodiment,” “some embodiments,” and so forth, means that a particular element (e.g., feature, structure, property, and/or characteristic) described in connection with the embodiment is included in at least one embodiment described herein, and may or may not be present in other embodiments, in addition, it is to be understood that the described element(s) may be combined in any suitable manner in the various embodiments.

Numerical values in the specification and paragraphs of this application, particularly as they relate to components of the liquid compositions, reflect average values for a composition that may contain individual components of different characteristics. Furthermore, unless indicated to the contrary, the numerical values should be understood to include numerical values which are the same when reduced to the same number of significant figures and numerical values which differ from the stated value by less than the experimental error of conventional measurement technique of the type described in the present application to determine the value.

### The Active Pharmaceutical Ingredient:

The active pharmaceutical ingredient of the liquid compositions of the present invention is selected from the group consisting of: acetaminophen, guaifenesin, pseudoephedrine HCl, dextromethorphan, and ibuprofen. Other pharmaceutical actives within the broader classes of these exemplary ingredients may also be used such as actives used in treating or reducing symptoms of cough, cold, fever, and/or flu. In a preferred embodiment, the active pharmaceutical ingredient comprises, or is solely, acetaminophen.

If acetaminophen is present it may be present in amounts of up to about, 3.5 wt % of the formulation. Preferably acetaminophen is present in amounts of about 1.6% to about 3.5% of the formulation. Most preferably acetaminophen is present in an amount of about 3.2% of the formulation.

In another embodiment if acetaminophen is present it is present in weight ratio of solvent to acetaminophen (solvent:acetaminophen) between about 5:1 and 25:1 (for example 9.2:1). In another embodiment the liquid composition comprises acetaminophen in an amount between about 0.33 wt % to about 6.66 wt % of the total composition.

If guaifenesin is present it may be present in amounts of up to about 300 milligrams per 5 ml of the excipient base. Preferably, guaifenesin is present in amounts of about 10 to about 300 milligrams per 5 ml of the excipient base. Most preferably, guaifenesin is present in amounts of about 100 to about 200 milligrams per unit dose of the excipient base.

If pseudoephedrine HCl is present it may be present in amounts of between about 10 and about 60 milligrams per unit close of the excipient base. Preferably, pseudoephedrine is present in amounts of about 30 to about 60 milligrams per unit dose of the excipient base.

If dextromethophan is present it may be present in amounts of between about 5 and about 30 milligrams per unit dose of the excipient base. Preferably, dextromethorphan is present in amounts of about 10 to about 30 milligrams per unit dose of the excipient base.

If ibuprofen is present it may be present in amounts of from about 50 to about 200 milligrams per 5 ml of the excipient base. Preferably, ibuprofen is present in amounts of about 100 to about 200 milligrams per unit dose of the excipient base.

### The Liquid Carrier System:

The liquid carrier system of the liquid compositions of the present invention includes a solvent system and also optionally, but preferably, includes water. The solvent system includes polyethylene glycol and a co-solvent selected from the group consisting of propylene glycol, glycerin, and a sugar alcohol (e.g. sorbitol, maltitol, xylitol, etc. which act as solvents, sorbitol being preferred), or similar co-solvents. The co-solvent most preferably comprises propylene glycol and glycerin. However, solely propylene glycol, glycerin, or sugar alcohol can make up the entirety of the co-solvent. In other embodiment, the co-solvent preferably comprises all three propylene glycol, glycerin, and sugar alcohol. Without being bound by a mechanism of intended action it is believed that propylene glycol concentration within the co-solvent can be reduced by increasing the concentration of the other co-solvents, and vice versa.

The present inventors have discovered and herein provide specific selections of liquid carrier components and ranges which provide for dissolution and stabilization of the active ingredients such that the composition is present as a single-phase at room temperature. In one embodiment, in 100 ml of the liquid carrier the following components are present:

(i) about 5 to about 20 grams of polyethylene glycol having a molecular weight of about 1000 to about 2000,
(ii) about 3 to about 7 ml of propylene glycol,
(iii) about 5 to about 13 ml of glycerin,
(iv) about 40 lo about 70 ml of liquid sugar,
(v) about 10 to about 20 ml of sorbitol solution, and
(vi) about 15 to about 35 ml water.

In another embodiment:

(i) the weight ratio of polyethylene glycol to propylene glycol is between about 3:1 and about 8:1.

In another embodiment, the liquid composition will include:

(i) Polyethylene glycol having molecular weight of 1300-1600, 15-50% by weight, particularly 15 to 20% (e.g., 18%);
(ii) Propylene glycol, 2-25% by weight particularly 2 to 8% (e.g., 5%);
(iii) Glycerin, 2-13% by weight, particularly 5 to 10% (e.g., 9%);
(iv) Sorbitol solution, 2%-20%, particularly 10 to 15% (e.g., 13%); and
(v) 0-50% by weight of water, particularly 15 to 35% (e.g., 15%).

As noted above, purified water may optionally be present in varying amounts and preferably makes up less than 50 wt % (for example between 30 to 40 wt %) of the liquid carrier system. In another embodiment, the water is preferably present in an amount not less than about 5-10 wt % of the composition.

The poly ethylene glycols useful in the practice of the present invention include those having an average molecular weight of about 1000 to about 2000. Preferably, the polyethylene glycol has an average molecular weight of about 1400 to about 1600. Most preferably, the polyethylene glycol used in the practice of the present invention has an average molecular weight of about 1450. The use of mixtures of such polyethylene glycols is further within the scope of the present invention. As stated above, the polyethylene glycol component may be present in amount of about 5 to about 20 grams, preferably about 15 to about 20 grams, per 100 milliliters of the liquid excipient base.

The polyethylene glycol in the specific examples is Polyethylene Glycol 1450 N.F. This polyethylene glycol, having a molecular weight of 1450 can be obtained from a number of suppliers. For instance, it is sold by Union Carbide Chemicals and Plastic Company, Inc. of Danbury, Conn. as CARBOWAX® 1450® and by Dow Chemical Company of Midland, Mich. as Dow Polyglycol E1450.

The propylene glycol is preferably a non-toxic grade of the compound and be pharmaceutically acceptable. Such pharmaceutically acceptable grades of propylene glycol are commercially available from, for example, The Dow Chemical Company.

Without being bound by a mechanism of action it is believed that polyethylene glycol when melted and mixed with a co-solvent selected from the group consisting of: propylene glycol, glycerine, and a sugar alcohol, serves to solubilize the pharmaceutical compounds and inhibit crystallization and degradation over time and/or at, below, or above, room temperature (about 20-25° C.). It is a further aspect of the present invention, that the formulations preferably remain suitable for any particular climate zone (e.g. I-IV), more preferably a combination of climate zones, and most preferably all climate zones. Further it is an aspect of the present invention that the compositions remain as a single phase solution for extended storage periods at depressed, normal and elevated temperatures. For example, the liquid composition is preferably present; as a single-phase after 6-months of storage at about 5° C.; as a single-phase after 6-months of storage at about 25° C.; and/or as a single-phase after 6-months of storage at about 40° C.

### Additive Agents:

In certain preferred embodiments the liquid composition will further comprises an additive selected from the group consisting of: a flavorant, a preservative, a pH buffer, and combinations thereof. Persons skilled in the art will quickly realize many other ingredients will be suitable for inclusion into the liquid compositions of the present invention. The liquid composition may further include suitable flavorants such as orange, grape, vanilla, cherry, cranberry, peppermint, spearmint, anise, blueberry raspberry, banana, chocolate, honey, caramel, citrus, strawberry, lemon, and lime. In preferred embodiments flavorants include cherry or grape. The addition of these type of flavorants is well known in the art and they are typically added such that the active flavor is present in the liquid composition in an amount corresponding to between about 0.01 wt % and about 1.0 wt % (e.g. between about 0.01 and about 0.12 wt %). In a further embodiment, the liquid composition comprises a sweetener including liquid sugar.

In another embodiment, the liquid composition comprises a pH buffer (e.g. a buffering agent) in an amount sufficient to maintain the pH of the liquid composition at a pH of less than 7.0 and more preferably in a range of between about 3.0 and 6.9, for example between 3.5 and 6.7, for example between 4 and 6.3 (e.g. between about 5.0 and 6.1, for example 5.9, 6.0, or 6.1). A non-limiting list of suitable buffers includes sodium citrate and citric acid.

In still a further embodiment, the liquid composition comprises a preservative agent. Preservatives useful in the present invention include but are not limited to sodium benzoate, sorbates, such as potassium sorbate, benzaldionium chloride and parabens (such as methyl, ethyl, propyl, and butyl p-hydroxybenzoic acid esters). Preservatives listed above are exemplary, but each preservative must be evaluated on an experimental basis based upon what is known in the art to one of ordinary skill in the art, in each formulation to assure compatibility and efficacy of the preservative. Methods for evaluating the efficacy of preservatives in pharmaceutical formulations are known to those skilled in the art. In one embodiment the preservative is a paraben such as methyl paraben, ethyl paraben, propyl paraben, and or butyl paraben.

Preservatives are generally present in amounts of up to about one gram per 100 ml of the pharmaceutical composition. Preferably the preservatives are present in amounts in the range of from about 0.01 w/v to about 0.4 w/v of the composition. Typically, the preservative methyl paraben and/or propyl paraben could be present in the range of about 0.1 w/v to about 0.2 w/v of the composition, for example. Preferably, methyl paraben could he present in a concentration of about 0.18 w/v while propyl paraben could be present in a concentration of about 0.02%.

In another embodiment, the liquid composition is essentially free of antioxidants (e.g. less than about 1 wt %, for example about 0.0 wt %). Oxidation of active ingredients is a common route to degradation of the active ingredient. In the present invention, stability of the active ingredients is imparted by the described selection of components of liquid carrier system which does not require the presence of an antioxidant.

A method of treating a Cold or Cold-Like Symptoms:

The liquid compositions of the present invention are preferred for use in treating or reducing cold or cold-like symptoms. A person suffering from cold or cold-like symptoms may find relief by orally ingesting a safe and effective amount of the liquid compositions as described above. The safe and effective amount of liquid composition is dependent upon the concentration of the therapeutic components present in the liquid compositions, the amount ingested, and the patient. In a preferred embodiment, the safe and effective amount of liquid composition is in a range between about 3.75 ml to 30 ml of the liquid composition. In other preferred embodiments the patient receiving a dosage of the composition is less than 12 years old, for example less than 5 years old (e.g. less than 2 or 1 years old).

In one embodiment, the safe and effective amount of liquid composition is measured in drops from a dropper, spoon, or syringe and may be less than 5 ml (e.g. 1-10 drops or 1-5 ml). For example where acetaminophen is included in the present formulation, a safe and effective dosage amount is as follows:

18-23 lbs (8.2 to 10.5 Kg)=120 mg−dose based upon 160 mg/5 ml is hence=3.75 ml syringe;

24-35 lbs (10.9 to 15.9 Kg)=160 mg=dose based upon 160 mg/5 ml=5 ml syringe.

A preferred liquid compositions is essentially free of ethanol (e.g. less than 2 wt % ethanol) and in most preferred embodiments less than 1 wt % (e.g. no ethanol or 0 wt % ethanol).

### Exemplary Liquid Composition I:

| | | | | Qty | |
|---|---|---|---|---|---|
| Item No | Ingredient Name | mg/5 ml | | (g)/batch | |
| 1 | Sugar Solution (IH) | 1348.684 | | 2157.89 | |
| 2 | Edetate Disodium (USP) | 5.000 | | 8.00 | |
| 3 | Methyl Paraben USP | 9.000 | | 14.40 | |
| 4 | Propyl Paraben USP | 1.000 | | 1.60 | |
| 5 | Polyethylene Glycol (NF) | 896.0531 | | 433.68 | |
| | 1450 | | | | |
| 6 | Glycerin (USP) 99% | 442.105 | | 707.37 | |
| 7 | Sodium Citrate (USP) | 10.500 | | 16.80 | |
| | Dihydrate | | | | |
| 8 | Acetaminophen (USP) | 160.000 | | 256.00 | |
| 9 | Propylene Glycol (USP) | 250.000 | | 400.00 | |
| 10 | Grape flavor (IH) | 11.513 | | 18.42 | |
| 11 | Sugar Solution (IH) | 1967.105 | | 3147.37 | |
| 12 | Sorbitol solution (USP) | 642.105 | | 1027.37 | |
| 13 | Citric Acid (USP) | 2.500 | | 4.00 | |
| | Anhydrous | | | | |
| 14 | Sucralose (NF) | 1.500 | | 2.40 | |
| 15 | Purified water (USP) | qs to 5 | ml | qs to 8 | L |
| 16 | Total volume | 5 | ml | 8 | L |

### Exemplary Liquid Composition II:

| Item | | | | Qty (g)/ | |
|---|---|---|---|---|---|
| No | Ingredient Name | mg/5 ml | | batch | |
| 1 | Sugar Solution (IH) | 1348.684 | | 2157.89 | |
| 2 | Edetate Disodium (USP) | 5.000 | | 8.00 | |
| 3 | Methyl Paraben (USP) | 9.000 | | 14.40 | |
| 4 | Propyl Paraben (USP) | 1.000 | | 1.60 | |
| 5 | Polyethylene Glycol (NF) | 896.053 | | 1433.68 | |
| | 1450 | | | | |
| 6 | Glycerin (USP) 99% | 442.105 | | 707.37 | |
| 7 | Sodium Citrate (USP) | 10.500 | | 16.80 | |
| | Dihydrate | | | | |
| 8 | Acetaminophen (USP) | 160.000 | | 256.00 | |
| 9 | Propylene Glycol (USP) | 156.250 | | 250.00 | |
| 10 | Cherry flavor (IH) | 7.500 | | 12.00 | |
| 11 | Sugar Solution (IH) | 1967.105 | | 3147.37 | |
| 12 | Sorbitol solution (USP) | 642.105 | | 1027.37 | |
| 13 | Citric Acid (USP) | 2.500 | | 4.00 | |
| | Anhydrous Fine Gran. | | | | |
| 14 | Sucralose (NF) | 1.500 | | 2.40 | |
| 15 | Purified water (USP) | Qs to 5 | ml | qs to 8 | L |
| 16 | Total volume | 5 | ml | 8 | L |

### Exemplary Liquid Composition III

This exemplary liquid formulation includes:

(i) Acetaminophen, about 1.6-3.5% by weight (e.g. about 3.2% by weight)
(ii) Polyethylene glycol having molecular weight of about 1300-1600 (e.g. 1400-1500, for example 1450), at 15-50% by weight (e.g. about 18%);
(iii) Propylene glycol about 3-7% by weight (e.g. about 5%),
(iv) Glycerin, about 5-13% by weight (e.g. about 9%);
(v) sorbitol solution, about 10%-20% (e.g. about 13%):
(vi) purified water, not less than 5-10% by weight (e.g., 15%); and
(vii) optional stabilizing and sweetening agents.

### Exemplary Method I of Producing A Liquid Composition:

The methods used to prepare-the liquid compositions of the present invention are not particularly limited. However, in a preferred embodiment the following steps can be used to prepare a flavored liquid composition including acetaminophen in a liquid carrier system comprising water and a solvent, wherein the liquid composition is present as a single phase at room temperature.

(I) Purified water is measured and introduced into a stainless steel vessel and heated between 50° C.-55° C.
(II) Liquid sugar is weighed and introduced to the vessel with stirring until a homogeneous solution is formed.
(III) Temperature is then preferably adjusted to 50-55° C. and edetate disodium, sodium citrate, citric acid, polyethylene glycol, are weighed and introduced to the vessel.
(IV) In separate vessel propylene glycol followed by methylparaben and of propylparaben under combined and stirred until a homogeneous mixture results.
(V) The contents of step (IV) are transferred to the vessel of step (III) mixed.
(VI) Glycerin is weighed and introduced to the vessel while mixed.
(VII) The temperature is reduced to below 35° C.
(VIII) Sucralose is weighed and introduced to the vessel.
(IX) Acetaminophen is weighed and added to the vessel and stirred until a single phase solution is present (e.g. 5 to 120 minutes).
(X) Flavor (e.g. grape, etc) is measured and added.
(XI) Optionally, additional liquid sugar is measured, added, and mixed.
(XII) Sorbitol solution is measured and added and mix for 5 minutes or more.
(XIII) Optionally, additional sodium citrate and citric acid can he measured, added, and mixed 5 minutes or more.
(XIV) Optionally add purified water to complete the formulation for scale up.
(XV) Screen the product through mesh screen.

### Exemplary Method II of Producing A Liquid Composition:

In another embodiment a method of preparing a liquid formulation, preferably containing acetaminophen and, optionally, one or more other active ingredients is provided wherein the liquid formulation is present as a single phase at room temperature. The method includes following steps: (a) solublizing sweetening agents in an aqueous vehicle (b) solublizing of preservative in propylene glycol (c) adding PEG and additional solvents selected from the group consisting of glycerin, sorbitol, and additional PG (d) adding and dissolving active pharmaceutical ingredient, (e) adjusting the volume to a standard volume by adding additional sweeteners, co-solvent, flavoring system, and/or water. The temperature of formulation is about 130±10° F. (e.g. 131±5° F).

### EXAMPLE

Having described the invention in detail, the following example is provided. The example should not be considered as limiting the scope of the invention, but merely as illustrative and representative thereof.

### Example

| Sr. No. | Ingredients | Qty for 100 ml solution |
|---|---|---|
| 1. | Acetaminophen | 3.20 gm |
| 2. | Propylene Glycol | 5.0 gm |
| 3. | Polyethylene Glycol 1450 | 17.92 gm |
| 4. | Glycerin | 8.84 gm |
| 5. | Sorbitol Solution | 12.84 gm |
| 6. | Sodium Citrate | Qs to pH 5.75 |
| 7. | Citric Acid | Qs to pH 5.75 |
| 8. | Purified water | qs |

Example 1 includes two separate batches (A and B) prepared at a pH of about 6.0. Both batches provided the following observations at the given stability conditions in sealed containers:

6 m 5° C.: Clear solution observed
6 m 25° C./60%: Clear solution observed
6 m 40° C./75%: Clear solution observed

Example 1 demonstrates that the formulations of the present invention are stable at various conditions. Minimal presence of PAP degradation was observed for both batches (A and B) as follows:

| Time/Temp/ | |
|---|---|
| Relative Humidity | % PAP Impurity Level measured using HPLC |
| Initial | 0.001 |
| 3 months 25° C./60% | 0.006 |
| 3 months 40° C./75% | 0.003 |

1. A liquid composition comprising an active pharmaceutical ingredient dissolved in a liquid carrier system, wherein the liquid carrier comprises polyethylene glycol and a co-solvent selected from the group consisting of: propylene glycol, glycerin, and a sugar alcohol, wherein the composition is essentially dye free, is present as a single-phase at room temperature, and optionally further comprises water.

2. The liquid composition of claim 1, wherein the liquid composition is essentially free of ethanol.

3. The liquid composition of claim 1, wherein the liquid composition is clear.

4. The liquid composition of claim 1, wherein the liquid composition is color-free.

5. The liquid composition of wherein the composition comprises:
(i) 15-50% by weight of polyethylene glycol having molecular weight of 1300-1600;
(ii) 2-25% by weight of propylene glycol;
(iii) 2-13% by weight of glycerin;
(iv) 2%-20% by weight of sorbitol solution; and
(v) 0-50% by weight of water.

6. The liquid composition of claim 5, wherein the composition comprises:
(i) 15-20% by weight of polyethylene glycol having molecular weight of 1400-1500;
(iii) 2 to 8% by weight of propylene glycol
(iii) 5-10% by weight of glycerin;
(iv) 1Q%-15% by weight of sorbitol solution; and
(v) 15-35% by weight of water.

7. The liquid composition of claim 6, wherein the composition comprises:
(i) 18% by weight of polyethylene glycol having molecular weight of 1450;
(iii) 5% by weight of propylene glycol
(iii) 9% by weight of glycerin;
(iv) 13% by weight of sorbitol solution; and
(v) 15% by weight of water.

8. The liquid composition of claim 5, wherein the active pharmaceutical ingredient is selected from the group consisting of: acetaminophen, guaifenesin, pseudoephedrine HCl, dextromethorphan, and ibuprofen.

9. The liquid composition of claim 8, further comprising an additive selected from the group consisting of: a flavorant, a preservative, and a pH buffer.

10. The liquid composition of claim 9, comprising a pH buffer, wherein the pH buffer is present in an amount sufficient to maintain the pH of the liquid composition in a range of between 3.0 and 6.9.

11. The liquid composition of claim 10, comprising a pH buffer, wherein the pH buffer is present in an amount sufficient to maintain the pH of the liquid composition in a range of between 5.0 and 6.1.

12. The liquid composition of claim 1, comprising a preservative selected from the group consisting of: methylparaben, ethylparaben, propylparaben, and butylparaben.

13. The liquid composition of claim 1, wherein the composition is present as a single-phase after 6-months of storage at about 25° C.

14. The liquid composition of claim 1, wherein the composition is present as a single-phase after 6-months of storage at about 25° C.

15. The liquid composition of claim 1, wherein the composition is present as a single-phase after 6-months of storage at about 40° C.

16-28. (canceled)

29. A method of treating a cold or cold-like symptoms in an individual, the method comprising administering to the individual a safe and effective amount of a liquid composition of claim 1.

30. (canceled)

31. The method of claim 29, wherein the individual is less than 2 years old.

32-33. (canceled)

34. A method of preparing a liquid formulation containing an active pharmaceutical ingredient, comprising the steps of:
(a) solublizing sweetening agents in an aqueous vehicle
(b) solublizing of preservative In propylene glycol
(c) adding of co-solvents comprising glycerin, sorbitol, and PEG, in aqueous vehicle
(d) adding and dissolving active pharmaceutical ingredient,
(e) adjusting the volume to a standard volume by adding additional sweeteners, co-solvents, flavoring system, and/or water.

35. The method of claim 34, wherein the active pharmaceutical ingredient comprises acetaminophen.

36. The liquid composition of claim 8, wherein the active pharmaceutical ingredient is acetaminophen.
